(19) United States
(12) Patent Application Publication
(10) Pub. No.: US 2021/0265010 A1
(43) Pub. Date: Aug. 26, 2021

(54) GENOTYPING DIPLOID SAMPLES WITH COVERAGE PLOT OF UNEXPLAINED READS

(71) Applicant: Sirona Genomics, Inc., Mountain View, CA (US)

(72) Inventors: Ming LI, Mountain View, CA (US); Chunlin WANG, Mountain View, CA (US)

(21) Appl. No.: 16/469,743

(22) PCT Filed: Dec. 15, 2017
(86) PCT No.: PCT/US17/66682

§ 371 Date: Jun. 14, 2019

Related U.S. Application Data
(60) Provisional application No. 62/434,900, filed on Dec. 15, 2016.

(51) Int. Cl. G16B 30/00; G16B 20/00

(52) U.S. Cl. CPC G16B 30/00; G16B 20/00

(57) ABSTRACT

The present disclosure relates to a method for the computation of the coverage of unexplained reads in the assignment of alleles for genetic analysis.

### FIELD

The present disclosure generally relates to the identification or alleles in a diploid genome.

### BACKGROUND

For diploid samples, there are two alleles present for each locus on the genome. If both alleles are the same, the locus is homozygous. Otherwise, the locus is heterozygous. When a locus is heterozygous, there exists a chance that typing software may only detect one allele and miss the other one. The coverage of the unexplained reads indicates a second allele present in this case.

The present disclosure provides the artisan with means to choose the correct second allele based on this information and thus obtain accurate genotype at this locus and significantly improves the accuracy of data analysis over existing technology.

### SUMMARY

One aspect of the present disclosure relates to a method for the computation of Coverage of Unexplained Reads (CUR) comprising the steps of: a) partitioning all the mapped reads into two sets, wherein the first set contains all the reads that can be mapped to the selected allele references and the second set contains the rest of the reads; b) computing the coverage at each position based on the second set of reads that cannot be mapped to selected alleles; and c) plotting the CUR in the coverage plot using bars, lines or symbols together with coverage of the selected alleles to determine if a real allele is missed and/or a wrong allele is selected.

In some embodiments, the present invention provides a method for computation of coverage of unexplained reads (CUR). Typically, such methods comprise obtaining sequence reads from a gene of interest and mapping the sequence reads to one or more reference allele sequences. After the reads are mapped, they are partitioned into two sets, the first set containing all the reads that can be mapped to the selected reference sequence and the second set containing the rest of the reads. This information is used to compute a coverage of unexplained reads (CUR) at each position based on the second set of reads that cannot be mapped to selected alleles. Such methods may also include determining whether the CUR is within the noise level of the target genomic region. In some embodiments, methods of the invention may be graphically represented, for example, CUR may be plotted in a coverage plot using bars, lines or symbols together with coverage of the selected alleles to determine if a real allele is missed and/or a wrong allele is selected. In some embodiments, the gene of interest is an HLA gene. In other embodiments, the gene of interest is not an HLA gene.

In some embodiments, the present invention provides a method for determining a haplotype of an HLA locus. Such methods typically comprise obtaining sequence reads from one or more HLA genes and mapping the sequence reads to one or more reference allele sequences. The mapped reads are then partitioned into two sets, the first set containing all the reads that can be mapped to the selected reference allele sequence and the second set containing the rest of the reads. A CUR may then be computed at each position based on the second set of reads that cannot be mapped to selected alleles. The haplotype of the HLA gene is determined to be that of the reference allele that results in the lowest CUR. In some embodiments, the CUR is reduced to the noise level.

### DETAILED DESCRIPTION

For purposes of explanation, specific nomenclature is set forth to provide a thorough understanding of the present disclosure. However, it will be apparent to one skilled in the art that these specific details are not required to practice the aspects of the present disclosure. Descriptions of specific applications are provided only as representative examples. The aspects of the present disclosure are not intended to be limited to the embodiments shown, but are to be accorded the widest possible scope consistent with the principles and features disclosed herein.

Sequencing reads are fragments of nucleotides that represent the sequence of one allele at particular region. Millions of overlapping reads can be generated to cover target regions on the genome by Next Generation Sequencing technology. During mapping analysis, each read can be compared to the reference sequences and aligned to the best matching sequence and position. The “read coverage” (also simply referred as “coverage” herein) at any position on the genome is defined as the number of overlapping reads covering that position after mapping. Normally coverage of a selected allele can be calculated from the reads mapped to the reference sequence for the selected allele. Here we define Coverage of Unexplained Reads (CUR, sometimes referred to as URC) as the coverage of all possible alleles for the locus minus the coverage of the selected alleles.

Traditional coverage measures the number of reads that are mapped to a selected allele reference. The Coverage of Unexplained Reads measures the number of reads that can NOT be mapped to the selected allele references. Traditional coverage can be determined and remain constant through the review. But the CUR is defined related to the selected alleles for the sample at particular locus, it changes with the selection of the alleles. When the correct alleles are selected, the CUR is reduced to the noise level, which provides a quality measure on the genotype calls.

By comparing the total sequence reads mapped to locus and the unique coverage of current predicted alleles, we are able to detect novel alleles, potential allele mistyping, which includes wrong allele and allele dropout. In addition, this method is able to detect cross-contamination, poor sequence run et al problem in the application of NGS shotgun sequencing technology for human leukocyte antigen (HLA) genotype.

When a majority of the reads can be mapped to the selected alleles, there is very low coverage of the unexplained reads.

We have found that the presently disclosed method provides a 1% improvement in accuracy, which surprisingly translates into an 83% reduction in read errors. This represents a drastic improvement over current methods and provides a significant clinical impact due to the ability to more accurately match alleles.

As used herein, the term “noise” relates to reads that are assigned to particular locus but inconsistent with the genotype of the sample. The noise reads can come from sequencing errors, sample contamination and other artifacts from the experiment. The coverage of all reads across a particular locus for a sample normally is above 200 folds or 200×. The coverage of noise reads has a normal range from 0 to 20×. The minimum coverage over the cDNA and genomic regions for an allele measures the quality of the genotype call. If the minimum coverage over cDNA or genomic regions is below 20× threshold, the genotype call has a low confidence.

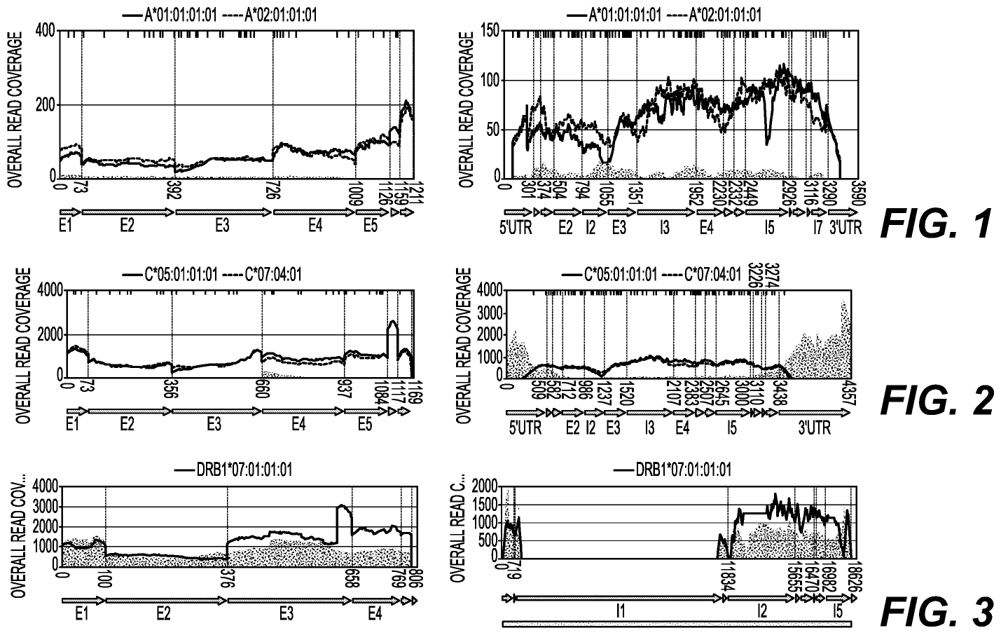

In FIG. 1, the left panel shows coverage along the cDNA reference sequences. The lines represent the coverage for the selected alleles for locus HLA-A, the shaded region shows the bar plot where each bar represents the Coverage of Unexplained Reads at one position. The right panel shows coverage along the genomic reference sequences. The red vertical bars above coverage curves indicate positions that are polymorphic between the selected alleles. The shaded region shows that CUR is very low compared to the coverage of selected alleles.

FIG. 2 represents a coverage plot of two correct alleles. The shadowed region indicates the difference between the total sequence reads mapped to locus and the unique coverage of selected alleles, which is correct in this case. The left panel shows the plot against cDNA reference sequences. The right panel shows the plot against genomic reference sequences.

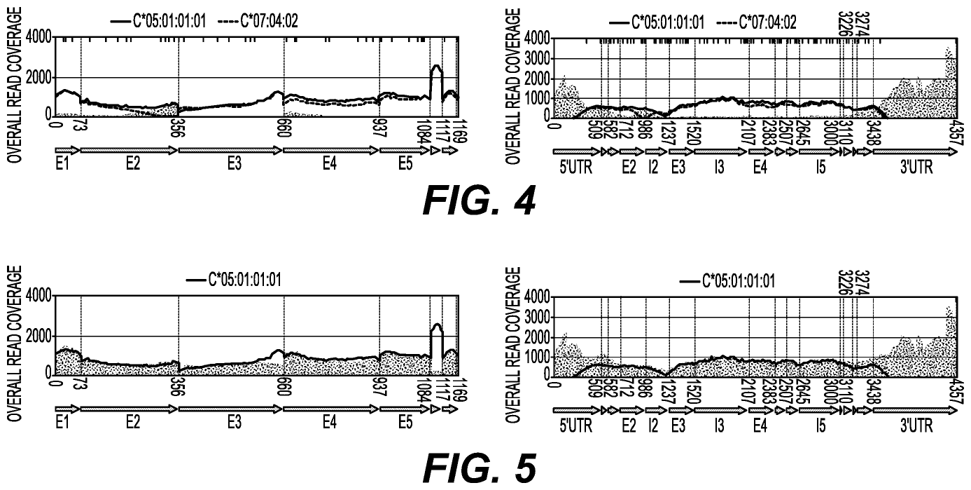

However, when a real allele is missed, elevated CUR is observed from the coverage plot. FIGS. 3 and 4 show examples. Using the methods of the present disclosure, the user can select the missed allele based on other quality metrics to reduce the CUR to a minimum level.

As shown in FIG. 3, when a real allele is missed in the genotype selection, coverage plot shows elevated shaded region. This indicates that significant amount of data cannot be explained by the selected allele.

FIG. 4 represents a coverage plot of two selected alleles where one is correct and one is incorrect. The shadowed area between 73-356 in left panel and center around 986 in right panel suggest that C*07:04:02 is not the right allele for this sample.

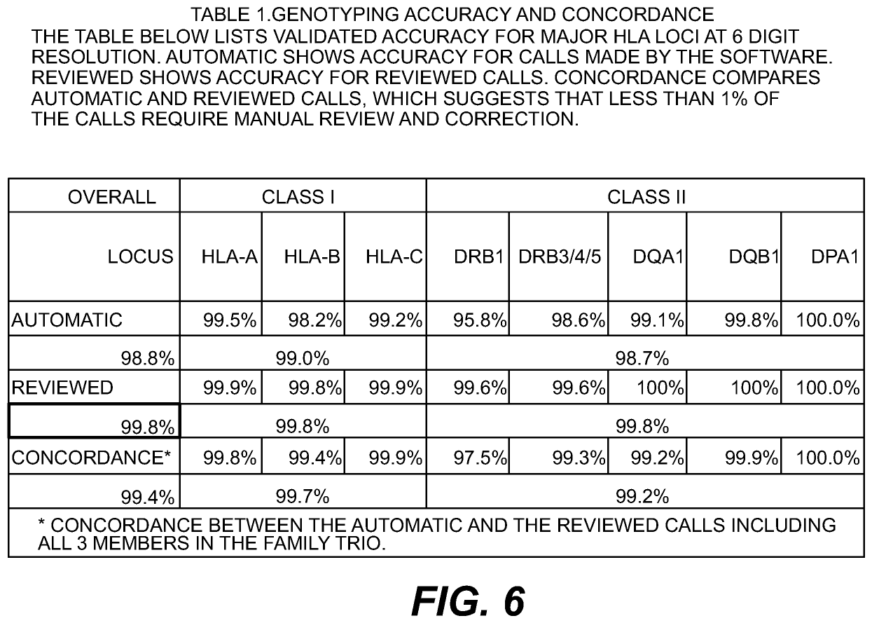

FIG. 5 depicts a coverage plot of a selected allele where one is missing. The shadowed area in both panels suggests that one allele is missing for this sample.

One aspect of the present disclosure relates to a method for the computation of CUR comprising the steps of: a) partitioning all the mapped reads into two sets, wherein the first set contains all the reads that can be mapped to the selected allele references and the second set contains the rest of the reads; b) computing the coverage at each position based on the second set of reads that cannot be mapped to selected alleles; and c) determining whether the CUR is within the noise level of the target genomic region.

In some embodiments, the method further comprises plotting the CUR in the coverage plot using bars, lines or symbols together with coverage of the selected alleles to determine if a real allele is missed and/or a wrong allele is selected.

In some embodiments, the method is employed for NGS HLA typing.

In another embodiment, the method is employed for genotyping alleles for any other diploid gene or target.

### Example 1

Samples of DNA comprising one or more genes of interest, for example genomic DNA comprising HLA genes, may be sequenced using standard techniques, for example, those found in US Patent publication no. 20140206547. In brief, PCR primers may be designed for each gene such that the most polymorphic exons and the intervening sequences may be amplified as a single product. If multiple genes are to be sequenced simultaneously, equimolar amounts of the PCR products may be pooled and ligated together to minimize bias in the representation of the ends of the amplified fragments. These ligated products may be randomly sheared to an average fragment size of 300-350 bp and prepared for sequencing, for example, using an Illumina sequencer (GAIIX,HiSeq2000, MiSeq, etc) according to the manufacturer's directions.

The sequences thus obtained may be aligned to genomic reference sequences. For HLA seqeuences, the sequences thus obtained may be aligned to sequences from the IMGT-HLA database with the NCBI BLASTN program. Over 20000 samples have been analyzed and reviewed with CUR. The accuracy of the genotyping results is assessed for both the automatic calls made by the software without incorporation of URC information and the reviewed calls that user corrected based on CUR information as shown in Table 1. The error rate is reduced by 83% with URC information through review.

The above description is for the purpose of teaching the person of ordinary skill in the art how to practice the claimed aspects of the disclosure and embodiments thereof, and it is not intended to detail all those obvious modifications and variations of it which will become apparent to the skilled worker upon reading the description. It is intended, however, that all such obvious modifications and variations be included within the scope of the present disclosure. The disclosure is intended to cover the components and steps in any sequence which is effective to meet the objectives there intended, unless the context specifically indicates the contrary. All patents and publications cited herein are entirely incorporated herein by reference.

What is claimed is:

1. A method for computation of coverage of unexplained reads (CUR) comprising the steps of:
a) obtaining sequence reads from a gene of interest;
b) mapping the sequence reads to one or more reference allele sequences;
c) partitioning all the mapped reads into two sets, wherein the first set contains all the reads that can be mapped to the selected reference sequence and the second set contains the rest of the reads; and
d) computing the CUR at each position based on the second set of reads that cannot be mapped to selected alleles.

2. A method according to claim 1, further comprising determining whether the CUR is within the noise level of the target genomic region.

3. A method according to claim 1, further comprising plotting the CUR in a coverage plot using bars, lines or symbols together with coverage of the selected alleles to determine if a real allele is missed and/or a wrong allele is selected.

4. A method according to claim 1, wherein the gene of interest is an HLA gene.

5. A method according to claim 1, wherein the gene of interest is not an HLA gene.

6. A method for determining a haplotype of an HLA locus, the method comprising:
a) obtaining sequence reads from one or more HLA genes;
b) mapping the sequence reads to one or more reference allele sequences;
c) partitioning all the mapped reads into two sets, wherein the first set contains all the reads that can be mapped to the selected reference allele sequence and the second set contains the rest of the reads;
d) computing the CUR at each position based on the second set of reads that cannot be mapped to selected alleles; and
determining the haplotype of the HLA gene wherein the haplotype is the allele that results in the lowest CUR.

7. A method according to claim 6, wherein the CUR is reduced to the noise level.
